(19) United States
(12) Patent Application Publication
(10) Pub. No.: US 2024/0272168 A1
(43) Pub. Date: Aug. 15, 2024

(54) IMMUNOSTAINING USING PROTEIN CONJUGATES

(71) Applicant: Advanced Biosystems, Inc., Menlo Park, CA (US)

(72) Inventors: Johann Kim, Redwood City, CA (US); Zuo-Rong Shi, Foster City, CA (US); Xu Chen, Bothell, WA (US); Grace Kim, Redwood City, CA (US)

(21) Appl. No.: 18/109,628

(22) Filed: Feb. 14, 2023

(51) Int. Cl. G01N 33/58

(52) U.S. Cl. CPC G01N 33/581; G01N 2333/908; G01N 2333/916; G01N 2474/20

(57) ABSTRACT

Provided herein are methods for detecting a target moiety in a biological sample suspected of containing the target moiety, by incubating the biological sample with an antibody and a fusion polypeptide, the fusion polypeptide comprising (i) an immunoglobulin-binding polypeptide and (ii) a reporting species.

### TECHNICAL FIELD

This document relates to methods for visualizing the location of one or more antigens within a preserved tissue sample.

### BACKGROUND

Immunohistochemistry (IHC) is a useful method for localizing a specific antigen to particular cell types in a heterogeneous population within a tissue, or to specific compartments within a cell. The sensitivity of IHC can be enhanced by amplification systems involving indirect detection of the primary antibody (Ab) by one or more additional steps. For example, the primary Ab can be detected either with secondary Ab labeled with an enzyme or fluorescent marker (two-step method). However, a limitation of indirect IHC is that primary Abs raised in a given species usually cannot be applied to tissues from that same species. In this situation, due to the homology of antigenic determinants on the targeting antibody and tissue, the secondary detecting antibody reacts with endogenous antigenic determinants in the tissue causing significant background staining, thereby reducing the usefulness of the assay for detecting the target antigen. For example, mouse monoclonal antibodies (MAbs), can be difficult to apply to the detection of antigens in mouse tissues by indirect methods due to the presence of endogenous mouse immunoglobulins (Igs) which are also recognized by the secondary Ab, creating background staining. Existing solutions to this challenge include, for example, applying Fab′ fragments to the tissue to block endogenous Igs before applying the secondary detection antibodies, to reduce background due to binding of secondary detection antibodies to endogenous Igs. However, this blocking method requires extended (e.g., hours to overnight) incubation periods which can slow down research and diagnostic laboratory pipelines and reduce throughput and efficiency.

### SUMMARY

The present disclosure is based, in part, on the discovery that immunoglobulin-binding proteins, for example Protein A, Protein G, Protein L, and recombinant modifications thereof, can be linked to a reporter and used for indirect detection of primary antibodies in IHC applications, for example, detection of an antigen in a biological sample. The compositions and methods disclosed herein do not require a blocking step after incubation of the primary antibody with the biological sample. Accordingly, the compositions and methods disclosed herein advantageously do not require any extended (e.g., hours to overnight) incubation steps, and thereby increase throughput and efficiency for laboratory IHC applications. The compositions and methods disclosed herein are advantageous for the detection of specific antigens when used as secondary detection in combination with primary Abs raised in a given species and applied to tissues from that same species. The compositions and methods of the present disclosure can be used for the detection of specific antigens in formalin-fixed paraffin-embedded tissues, wherein endogenous Igs have been denatured by the fixation process.

One aspect of the disclosure features a method for detecting a target moiety in a biological sample suspected of containing the target moiety, the method including incubating the biological sample with a first antibody such that the first antibody binds to the target moiety; incubating the biological sample and the first antibody bound to the target moiety with a fusion polypeptide, the fusion polypeptide including (i) an immunoglobulin-binding polypeptide and (ii) a reporting species, wherein the immunoglobulin-binding polypeptide is selected from the group consisting of Protein A, Protein G, Protein L, Protein A/G, Protein A/G/L, or variants thereof; visualizing the reporting species; and detecting, based on the location of the reporting species, the target moiety.

In some embodiments, the reporting species is a reporting enzyme. In some embodiments, the reporting species is a biotin molecule. In some embodiments, the method further includes, before the detecting step, incubating the biological sample with a reporting substrate.

In some embodiments, the reporting substrate includes an avidin molecule bound to a reporting enzyme. In some embodiments, the method further includes, before the detecting step, a chemical reaction between the reporting enzyme and the reporting substrate. In some embodiments, the immunoglobulin-binding polypeptide includes an amino acid sequence that is at least 80% identical to SEQ ID NO:1. In some embodiments, the immunoglobulin-binding polypeptide includes an amino acid sequence that is at least 90% identical to SEQ ID NO:1. In some embodiments, the immunoglobulin-binding polypeptide includes an amino acid sequence that is at least 95% identical to SEQ ID NO:1. In some embodiments, the immunoglobulin-binding polypeptide includes SEQ ID NO:1. In some embodiments, the immunoglobulin-binding polypeptide is Protein A. In some embodiments, the immunoglobulin-binding polypeptide is recombinant Protein A.

In some embodiments, the immunoglobulin-binding polypeptide comprises an amino acid sequence that is at least 80% identical to SEQ ID NO:2. In some embodiments, the immunoglobulin-binding polypeptide comprises an amino acid sequence that is at least 90% identical to SEQ ID NO:2. In some embodiments, the immunoglobulin-binding polypeptide comprises an amino acid sequence that is at least 95% identical to SEQ ID NO:2. In some embodiments, the immunoglobulin-binding polypeptide comprises SEQ ID NO:2. In some embodiments, the immunoglobulin-binding polypeptide is Protein G. In some embodiments, the immunoglobulin-binding polypeptide is recombinant Protein G.

In some embodiments, the immunoglobulin-binding polypeptide comprises an amino acid sequence that is at least 80% identical to SEQ ID NO:3. In some embodiments, the immunoglobulin-binding polypeptide comprises an amino acid sequence that is at least 90% identical to SEQ ID NO:3. In some embodiments, the immunoglobulin-binding polypeptide comprises an amino acid sequence that is at least 95% identical to SEQ ID NO:3. In some embodiments, the immunoglobulin-binding polypeptide comprises SEQ ID NO:3. In some embodiments, the immunoglobulin-binding polypeptide is Protein L. In some embodiments, the immunoglobulin-binding polypeptide is recombinant Protein L.

In some embodiments, the immunoglobulin-binding polypeptide comprises an amino acid sequence that is at least 80% identical to SEQ ID NO:4. In some embodiments, the immunoglobulin-binding polypeptide comprises an amino acid sequence that is at least 90% identical to SEQ ID NO:4. In some embodiments, the immunoglobulin-binding polypeptide comprises an amino acid sequence that is at least 95% identical to SEQ ID NO:4. In some embodiments, the immunoglobulin-binding polypeptide comprises SEQ ID NO:4. In some embodiments, the immunoglobulin-binding polypeptide is recombinant Protein A/G.

In some embodiments, the immunoglobulin-binding polypeptide comprises an amino acid sequence that is at least 80% identical to SEQ ID NO:5. In some embodiments, the immunoglobulin-binding polypeptide comprises an amino acid sequence that is at least 90% identical to SEQ ID NO:5. In some embodiments, the immunoglobulin-binding polypeptide comprises an amino acid sequence that is at least 95% identical to SEQ ID NO:5. In some embodiments, the immunoglobulin-binding polypeptide comprises SEQ ID NO:5. In some embodiments, the immunoglobulin-binding polypeptide is recombinant Protein A/G/L.

In some embodiments, the reporting enzyme is horseradish peroxidase. In some embodiments, the reporting enzyme is alkaline phosphatase. In some embodiments, the substrate is selected from the group consisting of ABTS, AEC, DAB, ECL, OPD, TMB, Homovanillic acid, Luminol, and AmplexRed. In some embodiments, the substrate is selected from the group consisting of Naphthol AS phosphate disodium salt; Naphthol AS-TR phosphate disodium salt; 6-Chloro-3-indoxyl phosphate, p-toluidine salt; 3-Indoxyl phosphate, bis(2-amino-2-methyl-1,3-propanediol) salt; 3-Indoxyl phosphate, disodium salt; and BCIP p-toluidine salt.

In some embodiments, the reporting species is a fluorescent protein. In some embodiments, the fluorescent protein is selected from the group consisting of GFP, RFP, YFP, or BFP. In some embodiments, the reporting species is a polypeptide conjugated to a fluorophore. In some embodiments, the biological sample is a tissue section from a mammal. In some embodiments, the biological sample is an FFPE tissue section from a mammal. In some embodiments, the mammal is a human. In some embodiments, the first antibody is a human monoclonal antibody.

In some embodiments, the mammal is a mouse. In some embodiments, the first antibody is a mouse monoclonal antibody. In some embodiments, the target moiety is an antigen. In some embodiments, the antigen is on the surface of a cell of the biological sample. In some embodiments, the antigen is localized to one or more organelles of a cell of the biological sample. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a schematic of the methods disclosed herein, targeting a mouse antigen (i.e., “Antigen X”) in mouse tissue using a mouse-raised primary antibody targeting “Antigen X.” As shown, enzyme-conjugated immunoglobulin-binding protein (e.g. Protein A/G) only binds to the primary antibody. Because endogenous Igs are denatured, the enzyme-conjugated immunoglobulin-binding protein does not non-specifically bind to endogenous Igs in the tissue, minimizing background signal.

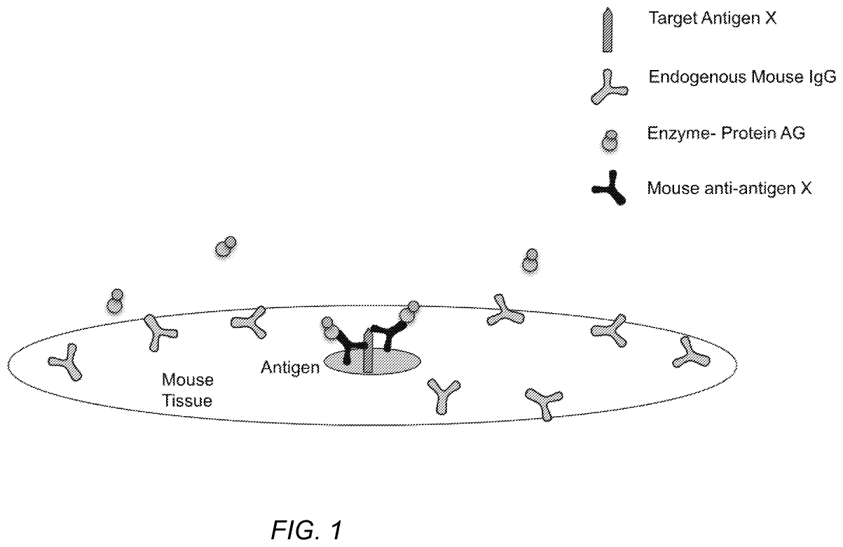

FIG. 2 is a table describing the relative affinities of binding to human and mouse immunoglobulins for Protein A, Protein G, Protein A/G, and Protein L. “+++” indicates relatively high affinity, “++” indicates medium affinity, “+” indicates relatively low affinity, and “−” indicates no binding.

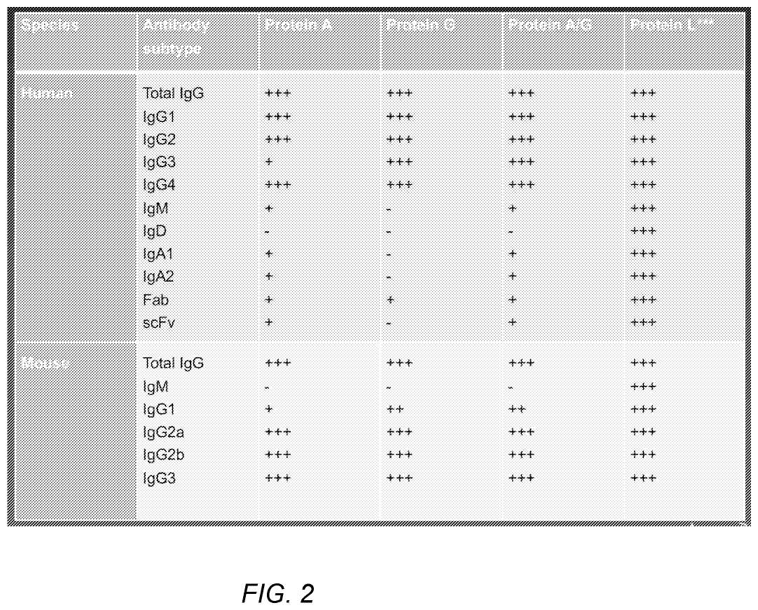

FIG. 3 is a table describing the relative affinities of binding to total IgG of rabbit, goat, sheep, guinea pig, hamster, donkey, pig, dog, cat, chicken, cow, and horse, for Protein A, Protein G, Protein A/G, and Protein L. “+++” indicates relatively high affinity, “++” indicates medium affinity, “+” indicates relatively low affinity, and “−” indicates no binding.

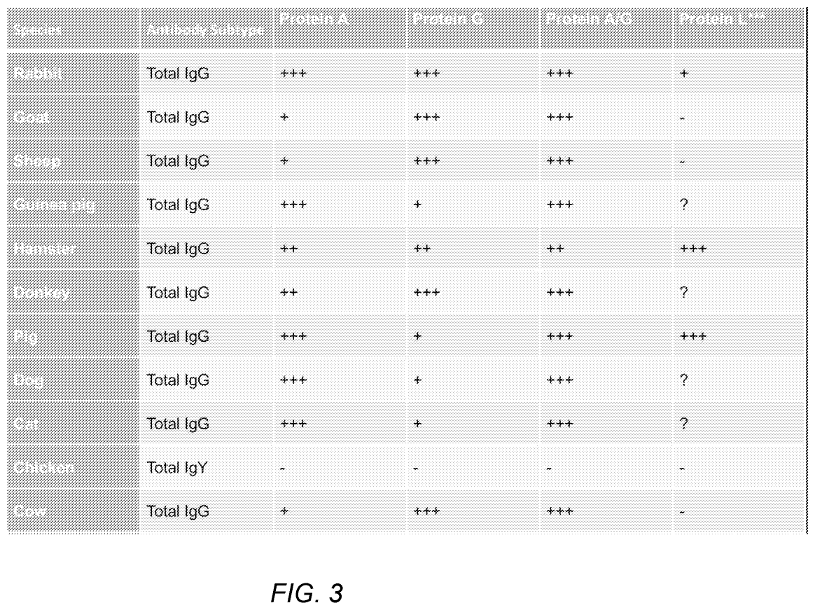

FIG. 4 is a series of light microscopy images comparing results of IHC methods using HRP-Protein A/G secondary detection of a mouse-on-mouse primary antibody (upper right) with prior art Fab′ fragment blocking methods (lower right). For reference an image with no primary antibody (upper left) is provided.

FIG. 5 is a series of light microscopy images comparing results of IHC methods using HRP-Protein A/G secondary detection of a mouse-on-mouse primary antibody (upper right) with prior art Fab′ fragment blocking methods (lower right). For reference an image with no primary antibody (upper left) is provided.

FIG. 6 is a series of light microscopy images comparing results of IHC methods using HRP-Protein A/G secondary detection of a mouse-on-mouse primary antibody (upper right) with prior art Fab′ fragment blocking methods (lower right). For reference an image with no primary antibody (upper left) is provided.

FIG. 7 is a series of light microscopy images comparing results of IHC methods using HRP-Protein A/G secondary detection of a mouse-on-mouse primary antibody (upper right) with HRP-Protein A/G/L secondary detection of a mouse-on-mouse primary antibody (lower right). For reference images with no primary antibody (upper left and lower left) are provided.

FIG. 8 is a series of light microscopy images comparing results of IHC methods using AP-Protein A/G/L (lower middle) and AP-Protein A/G (lower right) secondary detection of a mouse-on-mouse primary antibody to a Fab′ blocking method (lower left). The Fab′ blocking method (lower left) used a Goat anti-mouse secondary antibody conjugated to AP for secondary detection, while the AP-Protein A/G/L (lower middle) and AP-Protein A/G (lower right) used immunoglobulin binding proteins conjugated to AP for secondary detection and did not require Fab′ blocking. For reference, images with no primary antibody (upper left, upper middle, upper right) are provided.

### DETAILED DESCRIPTION

The methods disclosed herein address the challenge of non-specific binding between secondary detection method and endogenous Igs in the target by using an immunoglobulin-binding protein fused to a reporter as the secondary detection method. The immunoglobulin-binding protein can be, for example, Protein A, Protein G, Protein L, recombinant versions thereof, modified versions thereof, recombinant Protein A/G, or recombinant Protein A/G/L. In some embodiments, the immunoglobulin-binding protein of the compositions and methods disclosed herein are linked to a reporter. In some embodiments, the reporter is an enzyme that catalyzes a detectable reaction. In some embodiments, the reporter is a fluorescent reporter detectable by fluorescent microscopy. In some embodiments, the immunoglobulin-binding protein is biotinylated and detected by a biotin-avidin or biotin-streptavidin detection system.

Further, the methods disclosed herein address common disadvantages of prior immunohistochemistry protocols. Current protocols require extensive testing of primary and secondary antibody combinations in order to optimize signal to background (i.e., non-specific) fluorescence. Additionally, current protocols require long blocking incubation times in an attempt to decrease background (i.e., non-specific) signal. Current protocols are limited by the types and number of compatible primary/secondary antibody combinations. The methods disclosed herein, using the compositions disclosed herein, give very low non-specific staining/fluorescence. The methods disclosed herein, using the compositions disclosed herein, do not require blocking steps. The methods disclosed herein, using the compositions disclosed herein, can be applied to a number of different species with good results.

As discussed in further detail below, the immunoglobulin proteins disclosed herein bind to the Ig of the antigen-specific primary antibody. In some embodiments, the primary antibody of the present disclosure was raised in the same species as the species of the tissue comprising the antigen to be detected. For example, the compositions and methods disclosed herein can be used in combination with mouse-derived primary antibodies to detect antigens in mouse tissues. In some embodiments, the compositions and methods disclosed herein can be used in combination with human-derived primary antibodies to detect antigens in human tissues. In some embodiments, the compositions and methods disclosed herein can be used in combination with rat-derived primary antibodies to detect antigens in rat tissues. In some embodiments, the compositions and methods disclosed herein can be used in combination with monkey-derived primary antibodies to detect antigens in monkey tissues.

In some embodiments, the methods disclosed herein can be performed on formalin-fixed, paraffin-embedded (FFPE) tissue sections. During the fixation process of FFPE tissue sections, endogenous Igs are denatured, such that the immunoglobulin-binding protein of the compositions and methods disclosed herein, and linked reporter, do not non-specifically bind to endogenous Igs in the tissue, thereby eliminating background signal due to the presence of endogenous Igs. Rather than using a secondary antibody which can recognize both natural and denatured epitopes, and non-discriminately bind to both endogenous antibodies and the primary antibody used in the assay, the methods described herein use immunoglobulin binding proteins, e.g., Protein A, Protein G, Protein L, Protein A/G, Protein A/G/L, or recombinant variants thereof, that selectively recognize the primary antibodies used in the assay as the primary antibodies are not denatured.

### Methods

In some methods described herein, a immunoglobulin-binding protein fused to a reporter is used as a secondary layer of detection, wherein the immunoglobulin-binding protein portion of the composition specifically binds to a primary antibody that has bound to the antigen of interest within the tissue sample. First, a primary antibody can be applied to the tissue sample, and the primary antibody specifically binds the antigen of interest. Next, the immunoglobulin-binding protein fused to a reporter can be applied to the tissue sample, wherein the immunoglobulin-binding protein portion of the composition specifically binds to the primary antibody. After binding to the primary antibody that has bound to the antigen of interest, the reporter portion of the composition can be detected by one of several means of detection, as described herein. Detection of the reporter portion of the composition facilitates localization of the antigen within the tissue sample. In some embodiments, the primary antibody was raised in a given animal species and applied to tissues from that same animal species. In such cases, the immunoglobulin-binding protein portion of the composition specifically binds to the primary antibody, but not to endogenous Igs in the tissue sample. In such cases, a blocking step to block endogenous Igs is not required. As described in further detail below, a wide variety of antigens can be detected using the compositions and methods disclosed herein, including using a primary antibody that was raised in a given animal species and applied to tissues from that same animal species, while minimizing background signal without requiring blocking of endogenous Igs.

### Incubation

In some embodiments, the methods disclosed herein begins with antigen retrieval, which may vary in terms of reagents and methods. The antigen retrieval process can involve pressure cooking, protease treatment, microwaving, or heating histologic sections in baths of appropriate buffers, with the goal of unmasking antigens hidden by formalin crosslinks or other fixation. See, e.g., Leong et al. Appl. Immnunohistochem. 4(3): 201(1996). (3):201 (1996).

In some embodiments, the methods disclosed herein do not include a protein blocking step. In some embodiments, the methods disclosed herein do not include a Fab′ fragment blocking step. In some embodiments, the methods disclosed herein do not include an extended (e.g., hours to overnight) Fab′ fragment blocking step.

After antigen retrieval, the tissue section can be exposed to the desired primary antibody for a sufficient period of time and under suitable conditions to allow the primary antibody to bind to the target antigen in the tissue section. Appropriate conditions for achieving this can be determined by routine experimentation. The slide can then be washed to remove unbound and excess amounts of the primary antibody. In some embodiments, the tissue section is also counterstained to provide contrast to the detection of the target antigen of interest, or to identify morphological features of interest, such as cell membranes, nuclei and the like. Examples of nuclear counterstains that can be used include chromogenic stains, such as hematoxylin, nuclear fast red, methyl green, and fluorescent stains, such as Hoechst stain, DAPI and propidium iodide. Examples of other counterstains include eosin, which can be used to stain cytoplasm, and fluorophore-tagged phalloidin, for staining actin cytoskeleton in cells. The methods disclosed herein may be performed using an automated pathology system, which may include automated staining (conventional stains, histochemical techniques, immunostainers); automated in situ hybridization systems; automatic slide preparation (coverslip, slide drying) and integrated slide and cassette labeling. See, e.g., Roja et al., Review of imaging solutions for integrated, quantitative immunohistochemistry in the Pathology daily practice, Folia Histochemica et Cytobiologica, (2009) 47(3):349-354.

After exposure to the desired primary antibody, optional washing, and optional counterstain, in some embodiments, the tissue sample is exposed to the immunoglobulin-binding protein fused to a reporter. In some embodiments, the immunoglobulin-binding protein fused to a reporter is provided in an aqueous solution. In some embodiments, the aqueous solution includes, but is not limited to, phosphate-buffered saline (PBS), bovine serum albumin (BSA), proclin 300, Tween®20, dye, the immunoglobulin-binding protein fused to a reporter, and additional water. In some embodiments, the tissue is exposed to the aqueous solution providing the immunoglobulin-binding protein fused to a reporter at a concentration and for a period of time sufficient to allow binding of the immunoglobulin-binding protein to the primary antibody. In some embodiments, the tissue is exposed to the aqueous solution providing the immunoglobulin-binding protein fused to a reporter for about 1, 2, 3, 4, 5, 6, 7, 8, 9, 10, 11, 12, 13, 14, 15, 16, 17, 18, 19, or about 20 minutes. The slide can then be washed to remove any non-specifically bound immunoglobulin-binding protein. In some embodiments, a chromogenic substrate is contacted to the tissue sample. Example of enzymatic reporters and substrates are described in further detail below.

### Detection and Visualizing

After completing the staining process, the stained slide can be analyzed for location of the target antigen as provided by the detectable reporter, either by a human, e.g., a pathologist, or a computer programmed to detect the reporter fused to the immunoglobulin-binding protein. The analysis can be performed directly by viewing the slide through a microscope at low, medium (10-20×) and high power (40-60×), and is preferably performed by viewing one or more high resolution images of the slide taken at low or medium power. Low or medium power is typically used to detect stained neoplastic cells (both within and outside of tumor nests) and staining of associated stroma in the entire tissue section. These staining patterns are useful in defining the region of interest (ROI). Images taken at medium and high power can be used to examine individual tumor nests or other features of interest within a region of interest (ROI) if appropriate to increase the sensitivity of the analysis.

In embodiments, digital images may be obtained by any suitable means, including digital microscopy and digital slide scanning, as well as digital storage databases. Examples of commercially available slide scanning devices that will convert whole glass slides into digital images include the Aperio® family of eSlide capture devices, including the ScanScope FL (Aperio, Vista, Calif.); the Omnyx™ VL4 and V:120 Scanners (Pittsburgh, Pa.); and the iScan HT and iScan Coreo scanners from Ventana Medical Systems (Tucson, Ariz.).

In some embodiments, the digital image is analyzed using an automated, digital pathology system or an immunofluorescence pathology system which includes a processor that is configured to carry out any of the detection methods disclosed herein. Examples of commercially available image analysis software include: Matlab® with the Image Processing Toolbox™ by Math Works (Natick Mass.); PRECISION Image Analysis Solution (Aperio, Vista, Calif.), ImagePro® from Media Cyber netics (Rockville, Md.); Metamorph® Microscopy Automation & Image Analysis Software from Molecular Devices (Sunnyvale, Calif.); and Columbus™ Image Data Storage and Analysis System from PerkinElmer (Waltham, Mass.).

In some embodiments, to facilitate indirect detection, immunoglobulin-binding protein fused to a reporter as the secondary detection method is detected by, for example, fluorophores or an enzymatic reaction. Indirect detection according to the methods disclosed herein is advantageous for detection, for example, very low-expressed proteins because the signal can be amplified above the level of detection provided by primary detection.

In some embodiments, the immunoglobulin-binding protein fused to a reporter as the secondary detection method is detected by, for example, fluorophores or an enzymatic reaction. The detection method and available microscope determine the choice of label.

In some embodiments, for immunoglobulin-binding protein fused to fluorescent labels, detection is performed by fluorescent microscopy. Suitable fluorescent markers for fluorescent microscopy are described in further detail below. Especially for multi-color staining experiments, fluorescent labels can be advantageous for detecting different cellular compartments at the same time. A fluorescence microscope is an optical microscope that uses fluorescence instead of, or in addition to, scattering, reflection, and attenuation or absorption, to study the properties of organic or inorganic substances. In the context of the compositions and methods disclosed herein, a fluorescent microscope can be used to detect the immunoglobulin-binding protein fused to a fluorescent reporter, which has been bound to the target-bound primary antibody. In some embodiments, a fluorescence microscope can perform epifluorescence microscopy to detect the immunoglobulin-binding protein fused to a fluorescent reporter. In some embodiments, a fluorescence microscope can perform confocal microscopy, for example to perform optical sectioning to produce multi-layer resolution of the fluorescence image.

In some embodiments, non-fluorescent light microscopy can be used to detect the immunoglobulin-binding protein fused to an enzymatic reporter, wherein the enzymatic reporter catalyzes an enzymatic reaction at the site of the target that is detectable by light microscopy. In some embodiments, the catalysis of the reaction provides a colored product that is detectable by light microscopy. In some embodiments, for an enzymatic reporter, the enzyme is attached to the immunoglobulin-binding protein and then forms an insoluble colored product when an organic substrate is added. As discussed in further detail below, the enzymatic report can be, for example, horseradish peroxidase and alkaline phosphatase.

In some embodiments, the immunoglobulin-binding protein is labeled with a biotin molecule, i.e., biotinylated. In some embodiments, biotinylated immunoglobulin-binding proteins are detected by an avidin-biotin detection system. In some embodiments, the avidin-biotin detection system includes use of a chromogenic enzyme-substrate reaction, e.g., HRP or AP enzymes and their respective substrates. In some embodiments, a biotinylated detection enzyme (HRP or AP) is pre-incubated with free avidin to form large avidin-biotin-enzyme complexes for use in detecting the biotinylated immunoglobulin-binding protein. Typically, the avidin-biotinylated enzyme are mixed together in a specified ratio to prevent avidin saturation and incubated at room temperature to form the complex. An aliquot of this solution can then added to the tissue sample, and any remaining biotin-binding sites on the avidin bind to the biotinylated immunoglobulin-binding protein that is already bound to the primary antibody. The detection enzyme, e.g., HRP or AP, can then be detected by chromogenic reaction with a substrate.

In some embodiments, in order to further amplify the detection signal, a two-step system is used, wherein the tissue is exposed to the desired primary antibody, an immunoglobulin-binding protein is applied to the primary antibody-bound tissue, then an anti-immunoglobulin-binding protein secondary antibody conjugated to a reporter is applied to the tissue, thereby amplifying the detection signal. In some embodiments, the immunoglobulin-binding protein is Protein A, Protein G, Protein L, Protein A/G, or Protein A/G/L and the anti-immunoglobulin-binding protein secondary antibody is anti-Protein A, anti-Protein G, anti-Protein L, anti-Protein A/G, or anti-Protein A/G/L. In some embodiments, the two-step system includes use of a chromogenic enzyme-substrate reaction, e.g., HRP or AP enzymes and their respective substrates. For example, the anti-immunoglobulin-binding protein secondary antibody can be conjugated to HRP or AP. The detection enzyme, e.g., HRP or AP, can then be detected by chromogenic reaction with a substrate. In some embodiments, the anti-immunoglobulin-binding protein secondary antibody is fused to a fluorescent reporter. The fluorescent reporter can then be detected by, e.g., fluorescent microscopy.

### Tissues

Tissue or cell samples according to the compositions and methods disclosed herein may be prepared by a variety of methods known to those of ordinary skill in the art, depending on the type of sample and the assay format. In some embodiments, a tissue section can be mounted on a slide or other support after an incubation with immune-specific reagents. The subsequent steps of the method are then conducted after mounting. For example, for microscopic inspection in immunohistochemistry (IHC), samples can be comprised in a tissue section mounted on a suitable solid support. For the production of photomicrographs, sections comprising samples may be mounted on a glass slide or other planar support, to highlight by selective staining certain morphological indicators of disease states or detection of detectable targets.

In some embodiments, a sample may be collected from a subject, fixed, and exposed to, for example, antibodies which specifically bind to the detectable target of interest. Sample processing steps may include, for example, antigen retrieval, exposure to a primary antibody, washing, exposure to a immunoglobulin-binding protein fused to a reporter, washing, and visualization. Washing steps can be performed with any suitable buffer or solvent, e.g., phosphate-buffered saline, TRIS-buffered saline, distilled water. The wash buffer can optionally contain a detergent, e.g., TWEEN®20 or NP-40.

In some embodiments, tissues are formalin-fixed, paraffin-embedded tissues. In some embodiments, prior to embedding, tissues are fixed with formaldehyde. Tissue fixation preserves antigens and prevents the autolysis and necrosis of harvested tissues.

Tissue fixation can prevent the degradation of antigens, cells, and tissue. In some embodiments, solutions of 10% buffered formalin and 4% formaldehyde or paraformaldehyde are used as tissue fixatives. In some embodiments, the methods of the disclosure are not performed on un-fixed tissue. In some embodiments, the methods of the disclosure are not performed on fresh tissue. In some embodiments, the methods of the disclosure are not performed on fresh frozen tissue.

In some embodiments, tissues are embedded in paraffin. Embedding tissue can provide support during sectioning and can make sections more robust during subsequent steps of a protocol. In some embodiments, tissues are dehydrated and cleared before adding paraffin (pre-heated to 60° C.) and left overnight. In some embodiments, after fixation and microtome sectioning, tissue samples are affixed to a glass slide and covered with a coverslip and optionally a mounting medium. In some embodiments, the methods of the disclosure are not performed on paraffin-embedded tissue.

The tissue sample can come from any suitable source tissue or cells that may comprise that antigen of interest. In some embodiments, the tissue is mammalian tissue. In some embodiments, the tissue is insect tissue. In some embodiments, the tissue is bird, e.g., chicken, tissue. In some embodiments, the mammalian tissue is rabbit, goat, sheep, guinea pig, hamster, donkey, pig, dog, cat, mouse, rat, monkey, or human tissue. In some embodiments, the tissue is human tissue. In some embodiments, the tissue is mouse tissue. In some embodiments, the tissue is monkey tissue. In some embodiments, the tissue sample is suspected to comprise cancerous cells.

In some embodiments, the tissue comprises prostate, lung, pancreas, cervical, renal, salivary gland uterine, gastric, thyroid, sinus, middle and inner ear, adrenal glands, appendix, hematopoietic system, bones and joints, spinal cord, breast, cerebellum, connective and soft tissue, corpus uteri, esophagus, eye, nose, eyeball, fallopian tube, extrahepatic bile ducts, mouth, intrahepatic bile ducts, kidney, appendix, larynx, lip, liver, lung and bronchus, lymph node, cerebral, spinal, nasal cartilage, retina, oropharynx, endocrine glands, female genital, ovary, penis and scrotum, pituitary gland, pleura, rectum, renal pelvis, ureter, peritonem, salivary gland, skin, small intestine, testis, thymus, thyroid gland, tongue, unknown, urinary bladder, uterus, vagina, labia, or vulva tissue. In some embodiments, the sample comprises cells selected from the group consisting of adipose, adrenal cortex, adrenal gland, adrenal gland-medulla, appendix, bladder, blood, blood vessel, bone, bone cartilage, brain, breast, cartilage, cervix, colon, colon sigmoid, dendritic cells, skeletal muscle, endometrium, esophagus, fallopian tube, fibroblast, gallbladder, kidney, larynx, liver, lung, lymph node, melanocytes, mesothelial lining, myoepithelial cells, osteoblasts, ovary, pancreas, parotid, prostate, salivary gland, sinus tissue, skeletal muscle, skin, small intestine, smooth muscle, stomach, synovium, joint lining tissue, tendon, testis, thymus, thyroid, uterus, and uterus corpus.

### Compositions

In some compositions described herein, an immunoglobulin-binding protein selected from the group consisting of protein A,G, L, A/G, or A/G/L is fused to a reporter. The compositions described herein can recognize a primary antibody interacting with a target antigen to provide visualization of the primary antibody without additional lengthy blocking steps.

### Antigens

The compositions and methods disclosed herein can be applied to a variety of targets for detection. Any target which can be recognized by a suitable primary antibody and immunoglobulin-binding protein fused to a reporter is compatible with the compositions and methods disclosed herein. In some embodiments, the target comprises a protein, such as a glycoprotein or lipoprotein, phosphoprotein, methylated protein, or a protein fragment, a peptide, or a polypeptide. In some embodiments, the target comprises a nucleic acid segment. In some embodiments, the target comprises a nucleic acid analog segment. In some embodiments, the target may comprise a lipid, a glycolipid, a sugar, a polysaccharide, a starch; a salt, an ion, or one of a variety of other organic and Inorganic substances, any of which may be free in solution or bound to another substance. The target may be expressed on the surface of cells of the sample, e.g., such as on a membrane or interface. Alternatively, the target can be contained in the interior of the sample. In the case of a cell sample, for instance, an interior target may comprise a target located within the cell membrane, periplasmic space, cytoplasm, or nucleus, or within an intracellular compartment or organelle. Targets can also include viral particles, or portions thereof, e.g., a nucleic acid segment or a protein. The viral particle can be a free viral particle, i.e., not associated with any other molecule, or it may be associated with any sample described above. In some embodiments, the target may be an antigen. In some embodiments, the target antigen to be detected is a protein localized at or near the surface of a cell. In some embodiments, the target antigen to be detected is in the extracellular matrix of a cell. In some embodiments, the target antigen to be detected is within the cytosol of a cell. In some embodiments, the target antigen to be detected is within one or more organelles of a cell.

### Immunoglobulin-Binding Proteins

Protein A is a 42 kDa surface protein originally found in the cell wall of the bacteria Staphylococcus aureus, and is encoded by the spa gene. Protein A can be useful in a variety of biochemical research applications because of its ability to bind immunoglobulins (Igs). It is composed of five homologous Ig-binding domains that fold into a three-helix bundle. Each domain is able to bind proteins from a variety of mammalian species, most notably IgGs. Protein A binds the heavy chain within the Fc region of most immunoglobulins and also within the Fab region in the case of the human VH3 family. Protein A has a consensus polypeptide sequence below:

(SEQ ID NO: 1)
MKKKNIYSIRKLGVGIASVTLGTLLISGGVTPAANAAQHDEAQQNAFYQ
VLNMPNLNADQRNGFIQSLKDDPSQSANVLGEAQKLNDSQAPKADAQQN
NFNKDQQSAFYEILNMPNLNEAQRNGFIQSLKDDPSQSTNVLGEAKKLN
ESQAPKADNNENKEQQNAFYEILNMPNLNEEQRNGFIQSLKDDPSQSAN
LLSEAKKLNESQAPKADNKFNKEQQNAFYEILHLPNLNEEQRNGFIQSL
KDDPSQSANLLAEAKKLNDAQAPKADNKFNKEQQNAFYEILHLPNLTEE
QRNGFIQSLKDDPSVSKEILAEAKKLNDAQAPKEEDNNKPGKEDNNKPG
KEDNNKPGKEDNNKPGKEDNNKPGKEDGNKPGKEDNKKPGKEDGNKPGK
EDNKKPGKEDGNKPGKEDGNKPGKEDGNGVHVVKPGDTVNDIAKANGTT
ADKIAADNKLADKNMIKPGQELVVDKKQPANHADANKAQALPETGEENP
FIGTTVFGGLSLALGAALLAGRRREL

In some embodiments, the present disclosure provides an immunoglobulin-binding protein fused to a reporter, wherein the immunoglobulin-binding polypeptide comprises an amino acid sequence that is at least 80%, 90%, 95%, 96%, 97%, 98%, 99%, or 100% identical to the Protein A consensus polypeptide sequence of SEQ ID NO:1. In some embodiments, the present disclosure provides an immunoglobulin-binding protein fused to a reporter, wherein the immunoglobulin-binding polypeptide comprises an amino acid sequence that is at least 80%, 90%, 95%, 96%, 97%, 98%, 99%, or 100% similar to the Protein A consensus polypeptide sequence of SEQ ID NO:1. In some embodiments, the present disclosure provides an immunoglobulin-binding protein fused to a reporter, wherein the immunoglobulin-binding polypeptide comprises an amino acid sequence that is at least 80%, 90%, 95%, 96%, 97%, 98%, 99%, or 100% identical to at least a portion of the Protein A consensus polypeptide sequence of SEQ ID NO:1. In some embodiments, the present disclosure provides an immunoglobulin-binding protein fused to a reporter, wherein the immunoglobulin-binding polypeptide comprises an amino acid sequence that is at least 80%, 90%, 95%, 96%, 97%, 98%, 99%, or 100% similar to at least a portion of the Protein A consensus polypeptide sequence of SEQ ID NO:1.

Protein G is an immunoglobulin-binding protein naturally expressed in group C and G Streptococcal bacteria much like Protein A, but with differing binding specificities. Protein G is approximately 60 kDa, and like Protein A, can bind to the Fab and Fc region of Igs. Protein G has a consensus polypeptide sequence below:

(SEQ ID NO: 2)
MEKEKKVKYFLRKSAFGLASVSAAFLVGSTVFAVDSPIEDTPIIRNGGE
LTNLLGNSETTLALRNEESATADLTAAAVADTVAAAAAENAGAAAWEAA
AAADALAKAKADALKEFNKYGVSDYYKNLINNAKTVEGIKDLQAQVVES
AKKARISEATDGLSDFLKSQTPAEDTVKSIELAEAKVLANRELDKYGVS
DYHKNLINNAKTVEGVKELIDEILAALPKTDTYKLILNGKTLKGETTTE
AVDAATAEKVFKQYANDNGVDGEWTYDDATKTFTVTEKPEVIDASELTP
AVTTYKLVINGKTLKGETTTKAVDAETAEKAFKQYANDNGVDGVWTYDD
ATKTFTVTEMVTEVPGDAPTEPEKPEASIPLVPLTPATPIAKDDAKKDD
TKKEDAKKPEAKKDDAKKAETLPTTGEGSNPFFTAAALAVMAGAGALAV
ASKRKED

In some embodiments, the present disclosure provides an immunoglobulin-binding protein fused to a reporter, wherein the immunoglobulin-binding polypeptide comprises an amino acid sequence that is at least 80%, 90%, 95%, 96%, 97%, 98%, 99%, or 100% identical to the Protein G consensus polypeptide sequence of SEQ ID NO:2. In some embodiments, the present disclosure provides an immunoglobulin-binding protein fused to a reporter, wherein the immunoglobulin-binding polypeptide comprises an amino acid sequence that is at least 80%, 90%, 95%, 96%, 97%, 98%, 99%, or 100% similar to the Protein G consensus polypeptide sequence of SEQ ID NO:2. In some embodiments, the present disclosure provides an immunoglobulin-binding protein fused to a reporter, wherein the immunoglobulin-binding polypeptide comprises an amino acid sequence that is at least 80%, 90%, 95%, 96%, 97%, 98%, 99%, or 100% identical to at least a portion of the Protein G consensus polypeptide sequence of SEQ ID NO:2. In some embodiments, the present disclosure provides an immunoglobulin-binding protein fused to a reporter, wherein the immunoglobulin-binding polypeptide comprises an amino acid sequence that is at least 80%, 90%, 95%, 96%, 97%, 98%, 99%, or 100% similar to at least a portion of the Protein G consensus polypeptide sequence of SEQ ID NO:2.

Protein L is an immunoglobulin-binding protein naturally expressed in some bacteria much like Protein A and Protein G, but with differing binding specificities. Unlike Protein A and Protein G, which bind to the Fc region of immunoglobulins (antibodies), Protein L binds antibodies through light chain interactions. Since no part of the heavy chain is involved in the binding interaction, Protein L binds a wider range of antibody classes than Protein A or G. Protein L binds to representatives of all antibody classes, including IgG, IgM, IgA, IgE and IgD. Single chain variable fragments (scFv) and Fab fragments also bind to Protein L. Protein L has a consensus polypeptide sequence below:

(SEQ ID NO: 3)
KEETPETPETDSEEEVTIKANLIFANGSTQTAEFKGTFEKATSEAYAYAD
TLKKDNGEYTVDVADKGYTLNIKFAGKEKTPEEPKEEVTIKANLIYADGK
TQTAEFKGTFEEATAEAYRYADALKKDNGEYTVDVADKGYTLNIKFAGKE
KTPEEPKEEVTIKANLIYADGKTQTAEFKGTFEEATAEAYRYADLLAKEN
GKYTVDVADKGYTLNIKFAGKEKTPEEPKEEVTIKANLIYADGKTQTAEF
KGTFAEATAEAYRYADLLAKENGKYTADLEDGGYTINIRFAGKKVDEKPE
EKEQVTIKENIYFEDGTVQTATFKGTFAEATAEAYRYADLLSKEHGKYTA
DLEDGGYTINIRFAG

In some embodiments, the present disclosure provides an immunoglobulin-binding protein fused to a reporter, wherein the immunoglobulin-binding polypeptide comprises an amino acid sequence that is at least 80%, 90%, 95%, 96%, 97%, 98%, 99%, or 100% identical to the Protein L consensus polypeptide sequence of SEQ ID NO:3. In some embodiments, the present disclosure provides an immunoglobulin-binding protein fused to a reporter, wherein the immunoglobulin-binding polypeptide comprises an amino acid sequence that is at least 80%, 90%, 95%, 96%, 97%, 98%, 99%, or 100% similar to the Protein L consensus polypeptide sequence of SEQ ID NO:3. In some embodiments, the present disclosure provides an immunoglobulin-binding protein fused to a reporter, wherein the immunoglobulin-binding polypeptide comprises an amino acid sequence that is at least 80%, 90%, 95%, 96%, 97%, 98%, 99%, or 100% identical to at least a portion of the Protein L consensus polypeptide sequence of SEQ ID NO:3. In some embodiments, the present disclosure provides an immunoglobulin-binding protein fused to a reporter, wherein the immunoglobulin-binding polypeptide comprises an amino acid sequence that is at least 80%, 90%, 95%, 96%, 97%, 98%, 99%, or 100% similar to at least a portion of the Protein L consensus polypeptide sequence of SEQ ID NO:3.

In some embodiments, a Protein A/G fusion protein is used in the methods disclosed herein. Protein A/G is a recombinant fusion protein that combines IgG binding domains of both Protein A and Protein G. Protein A/G contains four Fc binding domains from Protein A and two from Protein G, yielding a final mass of approximately 50 kDa. The binding of Protein A/G is less pH-dependent than Protein A, but otherwise has the additive properties of Protein A and G.

Protein A/G binds to all subclasses of human IgG. In addition, it binds to IgA, IgE, IgM, and IgD. Protein A/G also binds to all subclasses of mouse IgG. This allows Protein A/G to be used for purification and detection of mouse monoclonal IgG antibodies. Protein A/G has a consensus polypeptide sequence below:

(SEQ ID NO: 4)
MGSSHHHHHHSSGLVPRGSHMASMTGGQQMGRDPAANAAQHDEAQQNAFY
QVLNMPNLNADQRNGFIQSLKDDPSQSANVLGEAQKLNDSQAPKADAQQN
NFNKDQQSAFYEILNMPNLNEAQRNGFIQSLKDDPSQSTNVLGEAKKLNE
SQAPKADNNFNKEQQNAFYEILNMPNLNEEQRNGFIQSLKDDPSQSANLL
SEAKKLNESQAPKADNKFNKEQQNAFYEILHLPNLNEEQRNGFIQSLKDD
PSQSANLLAEAKKLNDAQAPKADNKFNKEQQNAFYEILHLPNLTEEQRNG
FIQSLKDDPSVSKEILAEAKKLNDAQAPKEDPNSSSVDKLAAALETYKLI
LNGKTLKGETTTEAVDAATAEKVFKQYANDNGVDGEWTYDDATKTFTVTE
KPEVIDASELTPAVTTYKLVINGKTLKGETTTEAVDAATAEKVFKQYAND
NGVDGEWTYDDATKTFTVTEKPEVIDASELTPAVTTYKLVINGKTLKGET
TTKAVDAETAEKAFKQYANDNGVDGVWTYDDATKTFTVTE

In some embodiments, the present disclosure provides an immunoglobulin-binding protein fused to a reporter, wherein the immunoglobulin-binding polypeptide comprises an amino acid sequence that is at least 80%, 90%, 95%, 96%, 97%, 98%, 99%, or 100% identical to the Protein A/G consensus polypeptide sequence of SEQ ID NO:4. In some embodiments, the present disclosure provides an immunoglobulin-binding protein fused to a reporter, wherein the immunoglobulin-binding polypeptide comprises an amino acid sequence that is at least 80%, 90%, 95%, 96%, 97%, 98%, 99%, or 100% similar to the Protein A/G consensus polypeptide sequence of SEQ ID NO:4. In some embodiments, the present disclosure provides an immunoglobulin-binding protein fused to a reporter, wherein the immunoglobulin-binding polypeptide comprises an amino acid sequence that is at least 80%, 90%, 95%, 96%, 97%, 98%, 99%, or 100% identical to at least a portion of the Protein A/G consensus polypeptide sequence of SEQ ID NO:4. In some embodiments, the present disclosure provides an immunoglobulin-binding protein fused to a reporter, wherein the immunoglobulin-binding polypeptide comprises an amino acid sequence that is at least 80%, 90%, 95%, 96%, 97%, 98%, 99%, or 100% similar to at least a portion of the Protein A/G consensus polypeptide sequence of SEQ ID NO:4.

The recombinant Protein A/G/L is a genetically engineered protein which combines the IgG binding profiles of all Protein A, Protein G and Protein L. Protein A/G/L is a gene fusion product. Recombinant fusion protein A/G/L is comprised of 5 Ig-binding regions of protein L (B1-B2-B3-B4-B5), 5 IgG binding domains from Protein A (E-D-A-B-C) and 2 Ig-binding region of protein G (C1-C3). The recombinant Protein A/G/L is ideal for purification of monoclonal or polyclonal IgG antibodies. Protein A/G/L binds to IgG from humans, mice, rats, cows, goats, sheep, rabbits, guinea pigs, pigs, dogs and cats.

Recombinant Protein A/G/L produced in E. coli is a single non-glycosylated polypeptide chain. Protein A/G/L is comprised of 5 IgG-binding regions of Protein A (E-D-A-B-C), 2 of protein G (C1-C3) and 5 of Protein L (B1-B2-B3-B4-B5) containing 805 amino acids in total and having a molecular mass of approximately 89 kDa. The cell wall binding region, cell membrane binding region and albumin binding region have been eliminated from the recombinant Protein A/G/L to provide the maximum specific IgG binding. Protein A/G/L has a consensus polypeptide sequence below:

(SEQ ID NO: 5)
NAAQHDEAQQNAFYQVLNMPNLNADQRNGFIQSLKDDPSQSANVLGEAQK
LNDSQAPKADAQQNNFNKDQQSAFYEILNMPNLNEAQRNGFIQSLKDDPS
QSTNVLGEAKKLNESQAPKADNNFNKEQQNAFYEILNMPNLNEEQRNGFI
QSLKDDPSQSANLLSEAKKLNESQAPKADNKFNKEQQNAFYEILHLPNLN
EEQRNGFIQSLKDDPSQSANLLAEAKKLNDAQAPKADNKFNKEQQNAFYE
ILHLPNLTEEQRNGFIQSLKDDPSVSKEILAEAKKLNDAQAPKEEDSLEG
SGSGTYKLILNGKTLKGETTTEAVDAATAEKVFKQYANDNGVDGEWTYDD
ATKTFTVTEKPEVIDASELTPAVTTYKLVINGKTLKGETTTKAVDAETAE
KAFKQYANDNGVDGVWTYDDATKTFTVTEEPRARPGSGSGKEETPETPET
DSEEEVTIKANLIFANGSTQTAEFKGTFEKATSEAYAYADTLKKDNGEYT
VDVADKGYTLNIKFAGKEKTPEEPKEEVTIKANLIYADGKTQTAEFKGTF
EEATAEAYRYADALKKDNGEYTVDVADKGYTLNIKFAGKEKTPEEPKEEV
TIKANLIYADGKTQTAEFKGTFEEATAEAYRYADLLAKENGKYTVDVADK
GYTLNIKFAGKEKTPEEPKEEVTIKANLIYADGKTQTAEFKGTFAEATAE
AYRYADLLAKENGKYTADLEDGGYTINIRFAGKKVDEKPEEKEQVTIKEN
IYFEDGTVQTATFKGTFAEATAEAYRYADLLSKEHGKYTADLEDGGYTIN
IRFAG

In some embodiments, the present disclosure provides an immunoglobulin-binding protein fused to a reporter, wherein the immunoglobulin-binding polypeptide comprises an amino acid sequence that is at least 80%, 90%, 95%, 96%, 97%, 98%, 99%, or 100% identical to the Protein A/G/L consensus polypeptide sequence of SEQ ID NO:5. In some embodiments, the present disclosure provides an immunoglobulin-binding protein fused to a reporter, wherein the immunoglobulin-binding polypeptide comprises an amino acid sequence that is at least 80%, 90%, 95%, 96%, 97%, 98%, 99%, or 100% similar to the Protein A/G/L consensus polypeptide sequence of SEQ ID NO:5. In some embodiments, the present disclosure provides an immunoglobulin-binding protein fused to a reporter, wherein the immunoglobulin-binding polypeptide comprises an amino acid sequence that is at least 80%, 90%, 95%, 96%, 97%, 98%, 99%, or 100% identical to at least a portion of the Protein A/G/L consensus polypeptide sequence of SEQ ID NO:5. In some embodiments, the present disclosure provides an immunoglobulin-binding protein fused to a reporter, wherein the immunoglobulin-binding polypeptide comprises an amino acid sequence that is at least 80%, 90%, 95%, 96%, 97%, 98%, 99%, or 100% similar to at least a portion of the Protein A/G/L consensus polypeptide sequence of SEQ ID NO:5.

### Reporters

In some embodiments, an immunoglobulin-binding protein of the compositions and methods disclosed herein is conjugated to a reporting species. The reporting species can be a molecule, protein, or moiety that can be specifically detected by, e.g., microscopy to specifically identify the location of the primary antibody in the tissue. In some embodiments, the reporting species is a reporting enzyme. In some embodiments, the reporting species is a biotin molecule, i.e., the immunoglobulin-binding protein is biotinylated. In some embodiments, the reporting species is an anti-immunoglobulin-binding protein secondary antibody.

In some embodiments, the immunoglobulin-binding protein of the compositions and methods disclosed herein is conjugated to a reporting enzyme. In some embodiments, the reporting enzyme is an enzyme that catalyzes a chromogenic reaction that can facilitate detection of a target molecule. In some embodiments, the reporting enzyme can be, for example, horseradish peroxidase (HRP), alkaline phosphatase, beta-galactosidase (GAL), glucose-6-phosphate dehydrogenase, beta-N-acetylglucosaminidase, B-glucuronidase, invertase, xanthine oxidase, firefly luciferase, and glucose oxidase (GO).

In some embodiments, tyramide signal amplification is used to amplify the detected signal generated by the immunoglobulin-binding protein conjugated to a reporter, e.g., HRP. The tyramide signal amplification process includes the use of HRP to enzymatically convert fluorophore tyramides to bind tyrosine residues on and surrounding the protein epitope targeted by the primary antibody. As a controlled enzymatic reaction, tyramide signal amplification does not diffuse from the site of enzyme activity and therefore, can enhance spatial resolution of the detected signal. In some embodiments, the immunoglobulin-binding protein fusion protein compositions disclosed herein are used with poly-HRP-mediated tyramide signal amplification. In some embodiments, the methods disclosed herein include incubation with conjugated tyramides for 2-10 minutes and addition of stop solution to halt HRP activity once the specific signal is detected.

The enzyme horseradish peroxidase (HRP), found naturally in the roots of horseradish, is useful for a wide range of biochemistry applications. It is a metalloenzyme that catalyzes the oxidation of various organic substrates by hydrogen peroxide. HRP can be used, in combination with a chromogenic substrate, to detect the presence of a molecular target. In the context of the compositions and methods disclosed herein, HRP can be conjugated to an immunoglobulin-binding protein. In some embodiments, HRP is conjugated to Protein A, Protein G, Protein L, recombinant versions thereof, modified versions thereof, recombinant Protein A/G, or recombinant Protein A/G/L. In some embodiments, horseradish peroxidase substrates, e.g., reporting substrates, can include, but are not limited to, 3-ethyl-2-[(3-ethyl-6-sulfo-1,3-benzothiazol-2-ylidene)hydrazinylidene]-1,3-benzothiazole-6-sulfonic acid (ABTS), aminoethyl carbazole (AEC), 3,3′-diaminobenzidine (DAB), 4-chloro-1-napthol (4-CN), enhanced luminol-based chemiluminescent substrate (ECL), o-phenylenediamine dihydrochloride (OPD), 3,3′-5-5′-tetramethylbenzidine (TMB), Homovanillic acid, Luminol, or AmplexRed.

The enzyme alkaline phosphatase (AP), which can be isolated from calf intestine, is a 140-kDa enzyme that catalyzes the hydrolysis of phosphate groups from a substrate molecule, resulting in a colored product or the release of light as one product of the reaction to detect the presence of a molecular target. AP has optimal enzymatic activity at a basic pH (pH 8-10) and can be inhibited by cyanides, arsenate, inorganic phosphate and divalent cation chelators, such as EDTA. In the context of the compositions and methods disclosed herein, AP can be conjugated to an immunoglobulin-binding protein. In some embodiments, AP is conjugated to Protein A, Protein G, Protein L, recombinant versions thereof, modified versions thereof, recombinant Protein A/G, or recombinant Protein A/G/L. In some embodiments, alkaline phosphates substrates, e.g., reporting substrates, can include, but are not limited to, Naphthol AS phosphate disodium salt; Naphthol AS-TR phosphate disodium salt; 6-Chloro-3-indoxyl phosphate, p-toluidine salt; 3-Indoxyl phosphate, bis(2-amino-2-methyl-1,3-propanediol) salt; 3-Indoxyl phosphate, disodium salt; BCIP p-toluidine salt.

In some embodiments, an immunoglobulin-binding protein is fused to a fluorescent reporter. The fluorescent reporter can be, for example, a fluorescent protein fused to immunoglobulin-binding protein, a fluorophore attached to the immunoglobulin-binding protein, or a fluorescent dye attached to the immunoglobulin-binding protein. Any suitable detectable label of the immunoglobulin-binding protein can be used. In some embodiments, the detectable label is a fluorophore. For example, the fluorophore can be from a group that includes: Alexa Fluor® 350 (a fluorophore with an excitation wavelength of about 350 nm), Alexa Fluor® 430 (a fluorophore with an excitation wavelength of about 430 nm), Alexa Fluor® 488 (a fluorophore with an excitation wavelength of about 488 nm), Alexa Fluor® 532 (a fluorophore with an excitation wavelength of about 532 nm), Alexa Fluor® 546 (a fluorophore with an excitation wavelength of about 546 nm), Alexa Fluor® 555 (a fluorophore with an excitation wavelength of about 555 nm), 30 Alexa Fluor® 568 (a fluorophore with an excitation wavelength of about 568 nm), Alexa Fluor® 594 (a fluorophore with an excitation wavelength of about 594 nm), Alexa Fluor® 633 (a fluorophore with an excitation wavelength of about 633 nm), Alexa Fluor® 647 (a fluorophore with an excitation wavelength of about 647 nm), Alexa Fluor® 660 (a fluorophore with an excitation wavelength of about 660 nm), Alexa Fluor® 680 (a fluorophore with an excitation wavelength of about 680 nm), Alexa Fluor® 700 (a fluorophore with an excitation wavelength of about 700 nm), Alexa Fluor® 750 (a fluorophore with an excitation wavelength of about 750 nm), BFP (Blue Fluorescent Protein), SpectrumAqua® (fluorophore with excitation wavelength of about 433 nm), SpectrumGreen® #1 (fluorophore with excitation wavelength of about 497 nm), SpectrumGreen® #2 (fluorophore with excitation wavelength of about 509 nm), SpectrumOrange® (fluorophore with excitation wavelength of about 559 nm), SpectrumRed® (fluorophore with excitation wavelength of about 587 nm), SYTO® 11 (fluorophore with excitation wavelength of about 508 nm), SYTOR 13 (fluorophore with excitation wavelength of about 488 nm). SYTOR 17 (fluorophore with excitation wavelength of about 621 nm), SYTOR 45 (fluorophore with excitation wavelength of about 452 nm), SYTOX® Blue (fluorophore with excitation wavelength of about 445 nm), SYTOX® Green (fluorophore with excitation wavelength of about 504 nm), SYTOX® Orange (fluorophore with excitation wavelength of about 547 nm), 5-TAMRA (5-Carboxytetramethylrhodamine), Tetramethylrhodamine (TRITC), Texas Red®/Texas Red®-X (fluorophore with excitation wavelength of about 595 nm), Texas Red®-X (fluorophore with excitation wavelength of about 595 nm and NHS Ester modification), Thiadicarbocyanine, Thiazole Orange, Y66F, Y66H, Y66W, YFP (Yellow Fluorescent Protein), RFP (Red Fluorescent Protein).

In some embodiments, an immunoglobulin-binding protein is fused to a biotin molecule, i.e., biotinylated. In some embodiments, the biotinylated immunoglobulin-binding protein is biotinylated Protein A, Protein G, Protein L, Protein A/G, or Protein A/G/L. Biotin is a vitamin (i.e., Vitamin H, Vitamin B7, Coenzyme R) that is present in small amounts in all living cells and is critical for a number of biological processes including cell growth and the citric acid cycle. Biotin is abundant in certain plant and animal tissues such as corn kernels, egg yolk, brain, liver, and blood. The valeric acid side chain of the biotin molecule can be derivatized in order to incorporate various reactive groups that facilitate the addition of a biotin tag to other molecules. Because biotin is relatively small (244.3 Daltons), it can be conjugated to many proteins and other molecules without significantly altering their biological activity. The highly specific interaction of biotin-binding proteins with biotin makes it a useful tool in assay systems designed to detect and target biological analytes.

In some embodiments, biotinylated immunoglobulin-binding proteins are detected by an avidin-biotin detection system. Avidin is a 68 kDa tetrameric glycoprotein composed of four identical subunits. Each subunit is glycosylated, and contains one binding site for biotin. Streptavidin is a neutral bacterial analog of avidin which occurs in a non-glycosylated form. Due to its glycosylation state, non-specific binding of biotin by avidin has been observed. Streptavidin and avidin both possess great affinity for biotin, with which they form irreversible bonds. This binding can be exploited in a number of biochemical assays, particularly immunohistochemistry, as disclosed herein.

In some embodiments, biotinylated immunoglobulin-binding proteins are detected by a streptavidin-biotin detection system. Streptavidin is a 66 kDa protein purified from the bacterium Streptomyces avidinii. Streptavidin homo-tetramers have a high affinity for biotin. With a dissociation constant (Kd) on the order of ≈10⁻¹⁴ mol/L, the binding of biotin to streptavidin is one of the strongest non-covalent interactions known in nature. Streptavidin is useful in molecular biology, e.g., for IHC applications, due to the streptavidin-biotin complex's resistance to organic solvents, denaturants (e.g., guanidinium chloride), detergents (e.g., SDS, Triton X-100), proteolytic enzymes, and extremes of temperature and pH.

The affinity of avidin for biotin and streptavidin is one the strongest known non-covalent interactions of a protein and ligand (Ka=10¹⁵M⁻¹) and allows biotin-containing molecules in a complex mixture to be discretely bound with avidin conjugates. The bond formation between biotin and avidin is very rapid, and once formed, it is unaffected by extremes in pH, temperature, organic solvents and other denaturing agents. In some embodiments, a biotinylated detection enzyme (HRP or AP) is pre-incubated with free avidin to form large avidin-biotin-enzyme complexes. Typically, the avidin-biotinylated enzyme are mixed together in a specified ratio to prevent avidin saturation and incubated at room temperature to form the complex. An aliquot of this solution can then added to the tissue sample, and any remaining biotin-binding sites on the avidin bind to the biotinylated immunoglobulin-binding protein that is already bound to the primary antibody. The detection enzyme, e.g., HRP or AP, can then be detected by chromogenic reaction with a substrate.

In some embodiments, an anti-immunoglobulin-binding protein secondary antibody conjugated to a reporter is used to detect the immunoglobulin-binding protein bound to the primary antibody. This approach can be used to further amplify the detection signal of the immunoglobulin-binding protein bound to the primary antibody. In some embodiments, the immunoglobulin-binding protein is Protein A, Protein G, Protein L, Protein A/G, or Protein A/G/L and the anti-immunoglobulin-binding protein secondary antibody is anti-Protein A, anti-Protein G, anti-Protein L, anti-Protein A/G, or anti-Protein A/G/L. In some embodiments, the anti-immunoglobulin-binding protein secondary antibody is conjugated to a reporting enzyme, e.g., HRP or AP. In some embodiments, the anti-immunoglobulin-binding protein secondary antibody is conjugated to a biotin molecule, i.e., the anti-immunoglobulin-binding protein secondary antibody is biotinylated. In some embodiments, the anti-immunoglobulin-binding protein secondary antibody is conjugated to a fluorescent reporter.

In some embodiments, the reporter is conjugated to the immunoglobulin-binding protein. In some embodiments, the immunoglobulin-binding protein in conjugated to a reporting enzyme. The immunoglobulin-binding protein can be conjugated to the reporting enzyme by any means known to a person of ordinary skill in the art. In some embodiments, the immunoglobulin-binding protein in conjugated to a fluorophore. The immunoglobulin-binding protein can be conjugated to a fluorophore by any means known to a person of ordinary skill in the art.

### EXAMPLES

The invention is further described in the following examples, which do not limit the scope of the invention described in the claims.

### Example 1: Detecting Target Antigen with Protein A Conjugate

To demonstrate the detection of antigens in tissues using enzyme-conjugated protein A species, antigens in formalin-fixed paraffin embedded (FFPE) tissues were detected using an HRP-conjugated protein A/G/AG/AGL detection system.

Formalin-fixed paraffin-embedded (FFPE) mouse brain tissue sections mounted on glass slides were retrieved from storage. To deparaffinize, slides were placed in a first xylene bath for 5 minutes under a ventilated fume hood at room temperature. Slides were subsequently placed in a second xylene bath for and additional 5 minutes under the fume hood at room temperature. To rehydrate the tissue sections, slides were placed in a first 100% ethanol bath for 3 minutes under the hood at room temperature; a second 100% ethanol bath for 3 minutes under the hood at room temperature; a 95% ethanol bath for 3 minutes under a fume hood at room temperature; and an 80% ethanol bath for 3 minutes under a fume hood at room temperature. To rinse, slides were placed in a tap water bath and rinsed for 1 minute, replacing the water twice to repeat the rinsing step for a total of three times. To block endogenous peroxidase, slides were placed in a 3% hydrogen peroxide bath for 10 minutes and subsequently rinsed with distilled water.

“Formulation 1” solution was applied such that the tissue section is covered (approximately 200 μL for each slide) and incubated for 15 minutes at room temperature. The ImmmunoHistoprobe X solution comprised the follow components:

Components for “Formulation 1”

1X PBS, pH 7.2
1% Bovine Serum Albumin (BSA) (w/v)
0.05% Proclin 300
0.5% Tween 20
Red coloring
Protein A/G/L Polymer Detector
H₂O

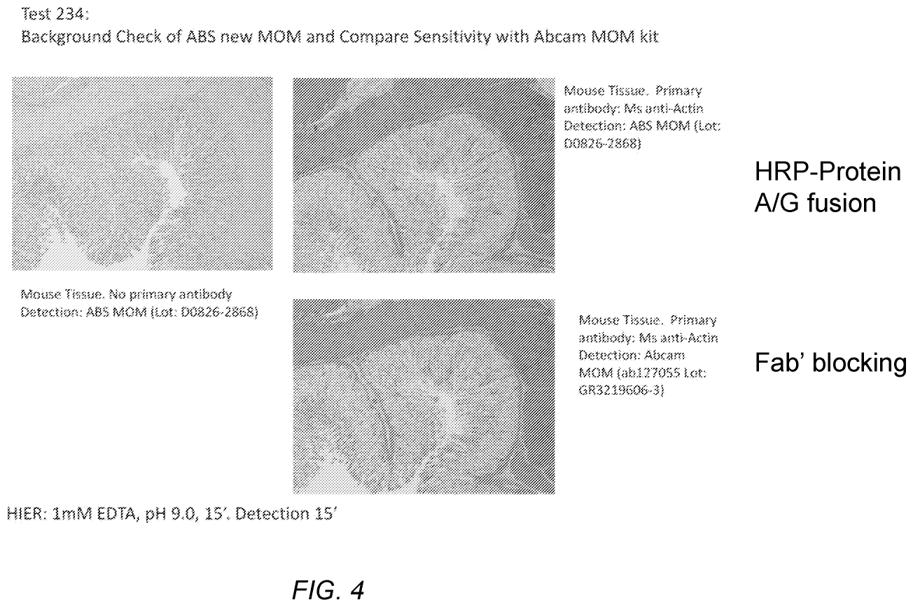

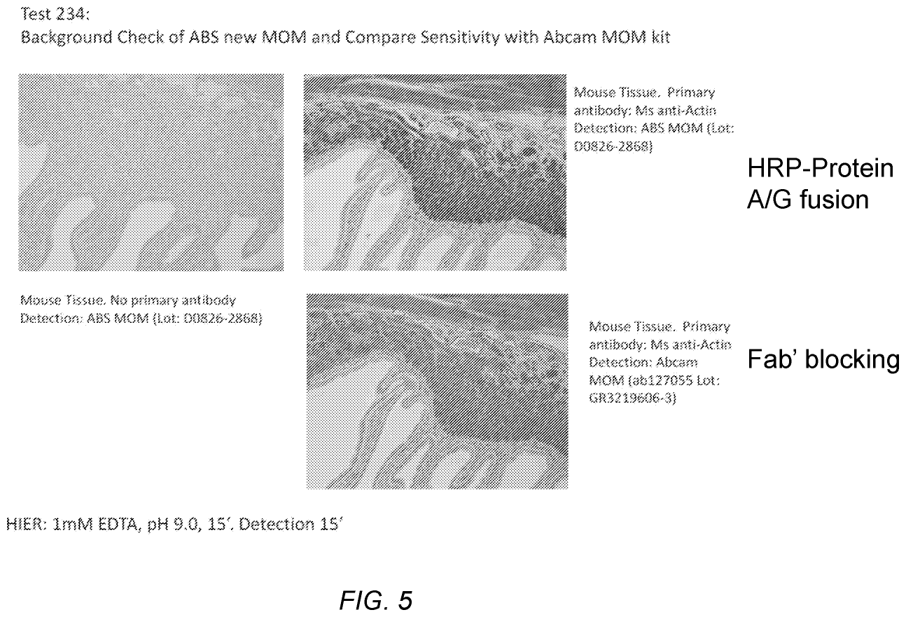

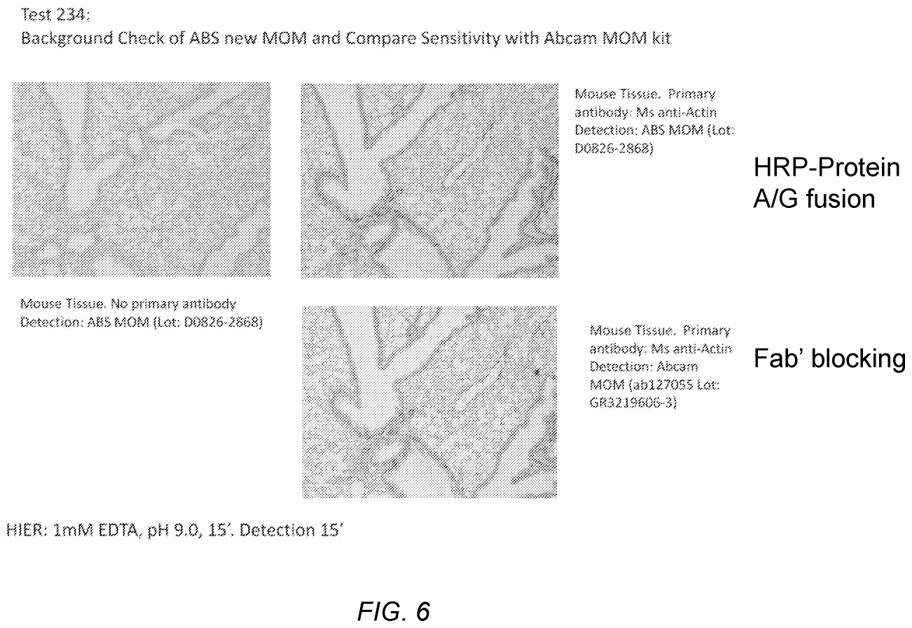

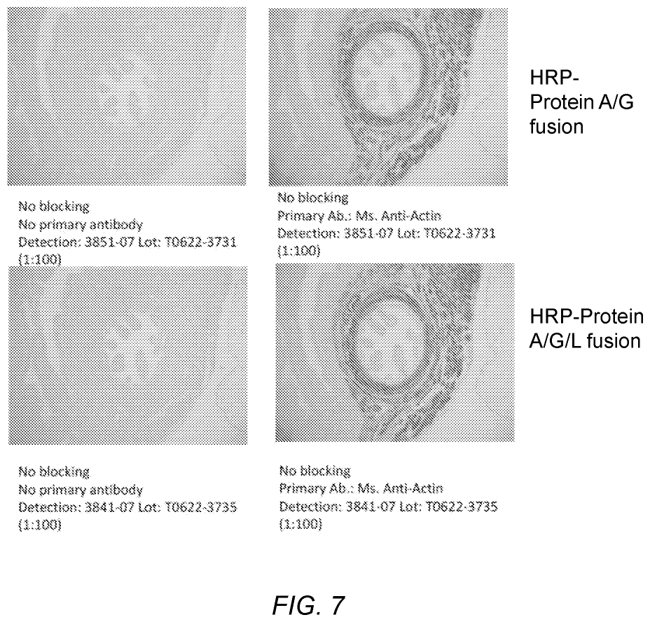

Next, the slides were washed three times with a wash buffer comprising 1× phosphate-buffered saline (pH 7.2) containing 0.05% Tween®20. 1-5 drops of DAB/AEC Chromogen Solution were added to each slide to cover the entire tissue section and incubated for 10 minutes. Colored precipitate was visualized at the sites of antigen expression as the chromogenic reporting substrate was converted by HRP reporting enzyme into insoluble end product. Slides were visualized with a light microscope. Representative images are provided in FIGS. 4-7.

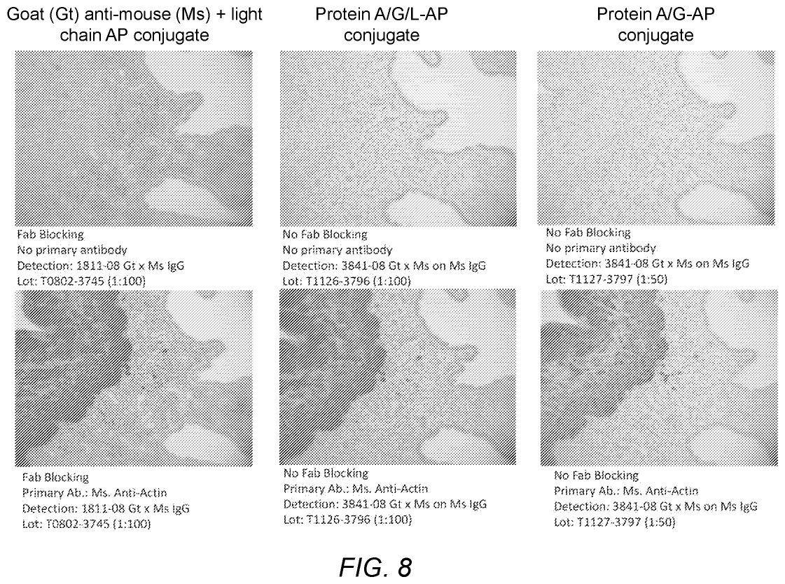

Further, antigens in formalin-fixed paraffin embedded (FFPE) tissues were detected using an AP-conjugated protein A/G/AG/AGL detection system. The same protocol was followed as for the HRP-conjugated protein A/G/AG/AGL detection system described above, using each of a Protein A/G/L-AP conjugate and a Protein A/G-AP conjugate to detect mouse actin in smooth muscle. FIG. 8 shows a series of light microscopy images comparing results of IHC methods using AP-Protein A/G/L (lower middle) and AP-Protein A/G (lower right) secondary detection of a mouse-on-mouse anti-actin primary antibody to a Fab blocking method (lower left). The Fab blocking method (lower left) used a Goat anti-mouse secondary antibody conjugated to AP for secondary detection, while the AP-Protein A/G/L (lower middle) and AP-Protein A/G (lower right) used immunoglobulin binding proteins conjugated to AP for secondary detection and did not require Fab blocking. For reference, images with no primary antibody (upper left, upper middle, upper right) are provided. These results indicate that an AP-conjugated protein A/G/AG/AGL detection system successfully detects a target antigen with similar results to a Fab blocking method, with minimal background signal.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for detecting a target moiety in a biological sample suspected of containing the target moiety, the method comprising:
incubating the biological sample with a first antibody such that the first antibody binds to the target moiety;
incubating the biological sample and the first antibody bound to the target moiety with a fusion polypeptide, the fusion polypeptide comprising (i) an immunoglobulin-binding polypeptide and (ii) a reporting species, wherein the immunoglobulin-binding polypeptide is selected from the group consisting of Protein A, Protein G, Protein L, Protein A/G, Protein A/G/L, or variants thereof;
visualizing the reporting species;
detecting, based on the location of the reporting species, the target moiety.

2. The method of claim 1, wherein the reporting species is a reporting enzyme.

3. The method of claim 1, wherein the reporting species is a biotin molecule.

4. The method of claim 1, wherein the method further comprises, before the detecting step, incubating the biological sample with a reporting substrate.

5. The method of claim 4, wherein the reporting substrate comprises an avidin molecule bound to a reporting enzyme.

6. The method of claim 5, wherein the method further comprises, before the detecting step, a chemical reaction between the reporting enzyme and the reporting substrate.

7. The method of claim 1, wherein the immunoglobulin-binding polypeptide comprises an amino acid sequence that is at least 80% identical to SEQ ID NO:1.

8. The method of claim 7, wherein the immunoglobulin-binding polypeptide comprises an amino acid sequence that is at least 90% identical to SEQ ID NO:1.

9. The method of claim 8, wherein the immunoglobulin-binding polypeptide comprises an amino acid sequence that is at least 95% identical to SEQ ID NO:1.

10. The method of claim 9, wherein the immunoglobulin-binding polypeptide comprises SEQ ID NO:1.

11. The method of claim 1, wherein the immunoglobulin-binding polypeptide is Protein A.

12. The method of claim 1, wherein the immunoglobulin-binding polypeptide is recombinant Protein A.

13. The method of claim 1, wherein the immunoglobulin-binding polypeptide comprises an amino acid sequence that is at least 80% identical to SEQ ID NO:2.

14. The method of claim 13, wherein the immunoglobulin-binding polypeptide comprises an amino acid sequence that is at least 90% identical to SEQ ID NO:2.

15. The method of claim 14, wherein the immunoglobulin-binding polypeptide comprises an amino acid sequence that is at least 95% identical to SEQ ID NO:2.

16. The method of claim 15, wherein the immunoglobulin-binding polypeptide comprises SEQ ID NO:2.

17. The method of claim 1, wherein the immunoglobulin-binding polypeptide is Protein G.

18. The method of claim 1, wherein the immunoglobulin-binding polypeptide is recombinant Protein G.

19. The method of claim 1, wherein the immunoglobulin-binding polypeptide comprises an amino acid sequence that is at least 80% identical to SEQ ID NO:3.

20. The method of claim 19, wherein the immunoglobulin-binding polypeptide comprises an amino acid sequence that is at least 90% identical to SEQ ID NO:3.

21. The method of claim 20, wherein the immunoglobulin-binding polypeptide comprises an amino acid sequence that is at least 95% identical to SEQ ID NO:3.

22. The method of claim 21, wherein the immunoglobulin-binding polypeptide comprises SEQ ID NO:3.

23. The method of claim 1, wherein the immunoglobulin-binding polypeptide is Protein L.

24. The method of claim 1, wherein the immunoglobulin-binding polypeptide is recombinant Protein L.

25. The method of claim 1, wherein the immunoglobulin-binding polypeptide comprises an amino acid sequence that is at least 80% identical to SEQ ID NO:4.

26. The method of claim 25, wherein the immunoglobulin-binding polypeptide comprises an amino acid sequence that is at least 90% identical to SEQ ID NO:4.

27. The method of claim 26, wherein the immunoglobulin-binding polypeptide comprises an amino acid sequence that is at least 95% identical to SEQ ID NO:4.

28. The method of claim 27, wherein the immunoglobulin-binding polypeptide comprises SEQ ID NO:4.

29. The method of claim 1, wherein the immunoglobulin-binding polypeptide is recombinant Protein A/G.

30. The method of claim 1, wherein the immunoglobulin-binding polypeptide comprises an amino acid sequence that is at least 80% identical to SEQ ID NO:5.

31. The method of claim 30, wherein the immunoglobulin-binding polypeptide comprises an amino acid sequence that is at least 90% identical to SEQ ID NO:5.

32. The method of claim 31, wherein the immunoglobulin-binding polypeptide comprises an amino acid sequence that is at least 95% identical to SEQ ID NO:5.

33. The method of claim 32, wherein the immunoglobulin-binding polypeptide comprises SEQ ID NO:5.

34. The method of claim 1, wherein the immunoglobulin-binding polypeptide is recombinant Protein A/G/L.

35. The method of claim 1, wherein the reporting enzyme is horseradish peroxidase.

36. The method of claim 1, wherein the reporting enzyme is alkaline phosphatase.

37. The method of claim 35, wherein the substrate is selected from the group consisting of ABTS, AEC, DAB, ECL, OPD, TMB, Homovanillic acid, Luminol, and AmplexRed.

38. The method of claim 36, wherein the substrates is selected from the group consisting of Naphthol AS phosphate disodium salt; Naphthol AS-TR phosphate disodium salt; 6-Chloro-3-indoxyl phosphate, p-toluidine salt; 3-Indoxyl phosphate, bis(2-amino-2-methyl-1,3-propanediol) salt; 3-Indoxyl phosphate, disodium salt; and BCIP p-toluidine salt.

39. The method of claim 1, wherein the reporting species is a fluorescent protein.

40. The method of claim 39, wherein the fluorescent protein is selected from the group consisting of GFP, RFP, YFP, or BFP.

41. The method of claim 1, wherein the reporting species is a polypeptide conjugated to a fluorophore.

42. The method of claim 1, wherein the biological sample is a tissue section from a mammal.

43. The method of claim 42, wherein the biological sample is an FFPE tissue section from a mammal.

44. The method of claim 43, wherein the mammal is a human.

45. The method of claim 44, wherein the first antibody is a human monoclonal antibody.

46. The method of claim 43, wherein the mammal is a mouse.

47. The method of claim 46, wherein the first antibody is a mouse monoclonal antibody.

48. The method of claim 1, wherein the target moiety is an antigen.

49. The method of claim 48, wherein the antigen is on the surface of a cell of the biological sample.

50. The method of claim 49, wherein the antigen is localized to one or more organelles of a cell of the biological sample.


SEQUENCE LISTING (5 sequences)

SEQ ID NO: 1 - protein, 516 aa, synthetic construct
MKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDNNKPGKEDNNKPGKEDNNKPGKEDNNKPGKEDGNKPGKEDNKKPGKEDGNKPGKEDNKKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 2 - protein, 448 aa, synthetic construct
MEKEKKVKYFLRKSAFGLASVSAAFLVGSTVFAVDSPIEDTPIIRNGGELTNLLGNSETTLALRNEESATADLTAAAVADTVAAAAAENAGAAAWEAAAAADALAKAKADALKEFNKYGVSDYYKNLINNAKTVEGIKDLQAQVVESAKKARISEATDGLSDFLKSQTPAEDTVKSIELAEAKVLANRELDKYGVSDYHKNLINNAKTVEGVKELIDEILAALPKTDTYKLILNGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEMVTEVPGDAPTEPEKPEASIPLVPLTPATPIAKDDAKKDDTKKEDAKKPEAKKDDAKKAETLPTTGEGSNPFFTAAALAVMAGAGALAVASKRKED

SEQ ID NO: 3 - protein, 365 aa, synthetic construct
KEETPETPETDSEEEVTIKANLIFANGSTQTAEFKGTFEKATSEAYAYADTLKKDNGEYTVDVADKGYTLNIKFAGKEKTPEEPKEEVTIKANLIYADGKTQTAEFKGTFEEATAEAYRYADALKKDNGEYTVDVADKGYTLNIKFAGKEKTPEEPKEEVTIKANLIYADGKTQTAEFKGTFEEATAEAYRYADLLAKENGKYTVDVADKGYTLNIKFAGKEKTPEEPKEEVTIKANLIYADGKTQTAEFKGTFAEATAEAYRYADLLAKENGKYTADLEDGGYTINIRFAGKKVDEKPEEKEQVTIKENIYFEDGTVQTATFKGTFAEATAEAYRYADLLSKEHGKYTADLEDGGYTINIRFAG

SEQ ID NO: 4 - protein, 540 aa, synthetic construct
MGSSHHHHHHSSGLVPRGSHMASMTGGQQMGRDPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEDPNSSSVDKLAAALETYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTE

SEQ ID NO: 5 - protein, 805 aa, synthetic construct
NAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDSLEGSGSGTYKLILNGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEEPRARPGSGSGKEETPETPETDSEEEVTIKANLIFANGSTQTAEFKGTFEKATSEAYAYADTLKKDNGEYTVDVADKGYTLNIKFAGKEKTPEEPKEEVTIKANLIYADGKTQTAEFKGTFEEATAEAYRYADALKKDNGEYTVDVADKGYTLNIKFAGKEKTPEEPKEEVTIKANLIYADGKTQTAEFKGTFEEATAEAYRYADLLAKENGKYTVDVADKGYTLNIKFAGKEKTPEEPKEEVTIKANLIYADGKTQTAEFKGTFAEATAEAYRYADLLAKENGKYTADLEDGGYTINIRFAGKKVDEKPEEKEQVTIKENIYFEDGTVQTATFKGTFAEATAEAYRYADLLSKEHGKYTADLEDGGYTINIRFAG